(19) United States
(12) Patent Application Publication
(10) Pub. No.: US 2023/0334380 A1
(43) Pub. Date: Oct. 19, 2023

(54) FUNCTION-INFORMED MATERIALS STRUCTURE

(71) Applicant: STOICHEIA, INC., Skokie, IL (US)

(72) Inventors: Andrey IVANKIN, Chicago, IL (US); Jordan H. SWISHER, Chicago, IL (US); Alexander P. MANTIS, Chicago, IL (US); Michael J. ASHLEY, Chicago, IL (US)

(21) Appl. No.: 18/298,093

(22) Filed: Apr. 10, 2023

Related U.S. Application Data
(60) Provisional application No. 63/329,326, filed on Apr. 8, 2022.

(51) Int. Cl. G06N 20/20

(52) U.S. Cl. CPC G06N 20/20

(57) ABSTRACT

Devices, systems, and methods within the present disclosure can assist in predicting material structural information based on functional characterization. Such predictions can be achieved by definition including a representation of a surface of a material as an ensemble of unit cells, determination of a pool of possible unit cells based on one or more material input properties, and computation of functional characteristics of the unit cells within the pool; and establishment including determination of a combination of unit cells from the pool to represent a potential surface structure of the material and computation of a corresponding cumulative functional characteristic for the material from the previously computed functional characteristics of individual unit cells of the combination, and validation of whether the computed cumulative functional characteristic matches at least one experimental measurement of the same functional property concerning the material. Iteration can assist.

### CROSS-REFERENCE

This Utility patent application claims the benefit of priority to U.S. Provisional Application No. 63/329,326, entitled FUNCTION-INFORMED MATERIALS STRUCTURES, filed on Apr. 8, 2022, the content of which is incorporated by reference herein in its entirety.

### FIELD

The present disclosure relates to devices, systems, and methods for material structures. More specifically, the present disclosure relates to devices, systems, and methods for material structures informed by function.

Exploring the hyperscale and multidimensional design space of complex polyelemental materials to find previously out-of-reach, disruptive catalysts can be extremely challenging, for example, with the combinatorial explosion of possibilities when considering combinations of constituent elements, such as stoichiometry, size, shape, and structure. An intelligent strategy that leverages the power of high-throughput experimentation, computation, and machine learning (ML) can assist in overcoming serendipity and/or increasing the success in materials discovery.

### SUMMARY

The present application discloses one or more of the features recited in the appended claims and/or the following features which, alone or in any combination, may comprise patentable subject matter.

According to an aspect of the present disclosure, a method of predicting material structural information based on functional characterization may include: (a) representing a surface of a material as an ensemble of unit cells; (b) determining a pool of possible unit cells based on one or more material input properties; (c) computing functional characteristics of the unit cells within the pool; (d) determining a combination of unit cells from the pool to represent a potential surface structure of the material and computing a corresponding cumulative functional characteristic for the material from the previously computed functional characteristics of individual unit cells of the combination; and (f) validating whether the computed cumulative functional characteristic matches at least one experimental measurement of the same functional property concerning the material.

According to another aspect of the present disclosure, a method of predicting material structural information based on functional characterization may include: (a) representing a surface of a material as an ensemble of unit cells; (b) determining a pool of possible unit cells based on one or more material input properties; (c) computing functional characteristics of the unit cells within the pool; (d) determining a combination of unit cells from the pool to represent a potential surface structure of the material and computing a corresponding cumulative functional characteristic for the material from the previously computed functional characteristics of individual unit cells of the combination; and (f) validating whether the computed cumulative functional characteristic matches at least one experimental measurement of the same functional property concerning the material. In some embodiments, the method may include (e) repeating the steps (a)-(f), concerning a material surface of at least one other material to generate a dataset comprising the cumulative functional characteristic for each validated material. The dataset may be configured for training a global machine learning algorithm for predicting surface configuration of unit cells for still another material based on one or more material input properties of the still another material.

In some embodiments, computing functional characteristics may include at least one of: determining reactant adsorption energies and associated current densities related to electrocatalysis, and determining electronic properties related to optical and/or magnetic characteristics of materials. In some embodiments, determining the functional characteristics of unit cells may be based on the density functional theory (DFT) calculations exclusively, or as a combination of DFT with machine learning.

In some embodiments, determining the combination of unit cells to represent a potential surface structure may be based on one or more of Monte Carlo simulations and a machine learning algorithm characterized as one or more of a deep learning model, a generative adversarial network (GAN), a transformer model, a reinforcement learning model, and an ensemble model. The machine learning model may include one of random forest and genetic algorithm.

In some embodiments, the global machine learning algorithm for predicting the surface structure may be based on one or more of a deep learning model, a generative adversarial network (GAN), a transformer model, a reinforcement learning model, and an ensemble model. The global machine learning model may include one of random forest and genetic algorithm.

In some embodiments, validating whether the computed functional property matches the at least one experimental measurement may include determining whether a prediction threshold is achieved. Determining whether the prediction threshold is achieved may include determining whether difference between the computed functional property and the experimentally measured functional properties is within a predetermined range of values. Validating may include determining that the computed functional property does not match the experimental measurements concerning the material surface of the material, and reiterating steps (a)-(f) until the potential surface structure yielding the computed functional property matches the at least one experimental measurement.

In some embodiments, configuration for training a machine learning algorithm may not require conducting steps (i)-(e) for the still another material. The one or more material input properties of the material may be defined only as composition of the material. The one or more material input properties of the still another material may be defined only as composition of the still another material.

In some embodiments, determining the combination of unit cells may include predicting the potential surface structure as a deterministic ensemble of unit cells. In some embodiments, determining the combination of unit cells may include predicting the potential surface structure as a probabilistic ensemble of unit cells.

According to another aspect of the present disclosure, a system may include at least one processor executing instructions stored in memory for conducting the methods recited above or below.

According to another aspect of the present disclosure, a method of predicting material structural information in relation to functional characterization may include determining functional characteristics of units cells among a pool of unit cells of a material surface; determining a predicted structure of the material surface based on the unit cell pool; and determining a predicted material activity based on the determined functional characteristics and the determined predicted structure, and outputting a characterization of material structural information of the material surface based on the predicted material activity.

In some embodiments, determining the predicted material activity may include determining whether a threshold prediction of material activity is achieved, and re-determining the predicted structure in response to determination that the threshold prediction has not be achieved. The threshold prediction of material activity may be determined by comparison of the predicted material activity with experimental results. Outputting may be performed in response to determination that the threshold prediction is achieved. Outputting a characterization may include a dataset for training a machine learning model for predicting the surface configuration of unit cells for new materials based on material input properties.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived. The emerging paradigm in heterogeneous catalysis suggests that the surface of complex catalysts features myriad different active sites which define the adsorption energies of reactants and intermediates, and, as such, activity and selectivity of the catalysts. The adsorption energies of active sites depend on their local electronic and geometric configurations and can be tuned. For example, such tuning may be achieved by mixing in new elements, changing size and shape, controlling crystal structure and/or oxidation state, and/or modifying the catalyst support, among other strategies.

### BRIEF DESCRIPTION OF DRAWINGS

The patent or application file contains at least one drawing executed in color. Copies of this patent or patent application publication with color drawings(s) will be provided by the Office upon request and payment of the necessary fee.

The detailed description particularly refers to the accompanying figures in which:

FIG. 1 is an illustration of comparison of predicted and experimental ORR activity trends for Ag—Ir—Pd—Pt—Ru MLs and Pt.

FIG. 2 is an illustration of a crystal-structure phase mapping as a demixing task wherein a phase diagram is inferred from a set of XRD patterns in a materials composition space (a), requiring identification of pure-phase prototypes and their composition-dependent modification. The input (a,b) and output (c-f) are illustrated for pattern #73 (d), with each demixed pattern shown in (c).

FIG. 3 is a workflow diagram example to determine the correct combination of unit cells with the known composition, size, and experimental results. Unit cells can be generated from the composition and DFT simulations can be run for each unit cell. The model can iteratively explores combinations of the unit cells and can compare the predicted activity (based on the DFT simulations) with the experimental results. Once a suitable threshold has been reached, the predicted structure can be paired with the input data.

### DETAILED DESCRIPTION

For the purposes of promoting an understanding of the principles of the disclosure, reference will now be made to a number of illustrative embodiments illustrated in the drawings and specific language will be used to describe the same.

The optimal adsorption energy of a (perfect) catalytic site toward crucial reaction intermediates must be neither “too strong” nor “too weak”. If intermediate molecules bind too strongly to the surface, they can poison the catalyst. If they bind too weakly, the intermediates can be released from the active sites prematurely, such as before undergoing subsequent transformation, typically via electron or proton transfer. By optimizing the combination and number of optimal active sites for key reaction intermediates, one can increase the activity and/or selectivity of a catalyst toward a desired chemical transformation.

The active sites framework can provide a way to computationally describe catalytic processes and guide the discovery of new catalysts. However, ab initio simulations often require the knowledge of the surface structure and therefore can be applicable to a very limited subset of materials that have already been synthesized and characterized. Traditional experimental characterization of the surface structure of complex catalysts can be extremely slow and/or can require state-of-the-art, expensive instrumentation. Expanding ab initio efforts to novel and not-yet-characterized materials can benefit from first predicting the likely structure for each material that presents a computationally-costly global optimization problem.

These issues can be complicated further by the fact that the surface structure of a catalyst is often dynamic and can evolve in the course of a reaction, for example, to a new quasi-steady state, distinct from the initial configuration. Therefore, it can be insufficient to know a surface structure of a virgin catalyst for purposes of accurately predicting its catalytic performance using ab initio simulations, but instead the knowledge of the operando structure is necessary. Unfortunately, not only do operando structures represent a tiny fraction of an already very limited number of structures characterized, but their characterization can be technologically more challenging. Devices, systems, and methods within the present disclosure concern approaches for uncovering the operando configuration of active sites on the surface of a catalyst based on high-throughput experimental functional characterization of catalysts, computational modeling, and/or machine learning (ML) that can avoid the need for prohibitively slow and/or expensive experimental structural analysis.

First-principles density functional theory (DFT) calculations have been widely used to compute adsorption energies of reactants onto the catalyst surface. However, performing one large calculation for all exposed atoms and nearest neighbors in a catalyst can be computationally intensive, and may not be feasible for a high-throughput workflow. Instead, the catalyst surface can be represented with a combination of smaller, e.g., 4×4×2 atom unit cells. Possible unit cells for a given composition of a catalyst can be constructed by allocating a random choice from the constituent elements to each lattice position.

Once the ensemble of possible unit cells for a catalyst is established, corresponding reactant adsorption energies can be calculated using DFT either for the entire set or a random subset of unit cells and the remaining adsorption energies can be later predicted using ML, or by combinations of direct and ML determination. Adsorption energies in turn relate to the current density and therefore electrocatalytic activity of the unit cell.

For example, Pedersen et al. previously showed that the current ji at a surface site i can be modeled using the Koutecky-Levich equation (1) with jD accounting for the diffusion-limited current and jk,i representing the kinetically-limited current (2).

$$\frac{1}{{j}_{i}} = \frac{1}{{j}_{D}} + \frac{1}{{j}_{k , j}} \qquad ( 1 )$$

$${j}_{{k}_{i}} = exp ( \frac{- \vert Δ {E}_{i} - Δ {E}_{opt} \vert + Δ {E}_{opt} - eU}{{k}_{B} T} ) \qquad ( 2 )$$

Here ΔE is the reactant's adsorption free energy and ΔEₒₚₜ is the optimal reactant's adsorption free energy given by the Sabatier principle, e is the elementary charge, U is the applied potential vs. reversible hydrogen electrode (RHE), k_{B} is the Boltzmann constant, and Tis the absolute temperature. The total current density for a unit cell is then a sum of current densities over all active sites, N, on the surface of the unit cell (3), and the total current density for a catalyst is a sum of current densities over all unit cells weighted by the probability of finding each type of the unit cell.

$${j}_{tot} = {{\sum }_{i}}^{N} {j}_{i} \qquad ( 3 )$$

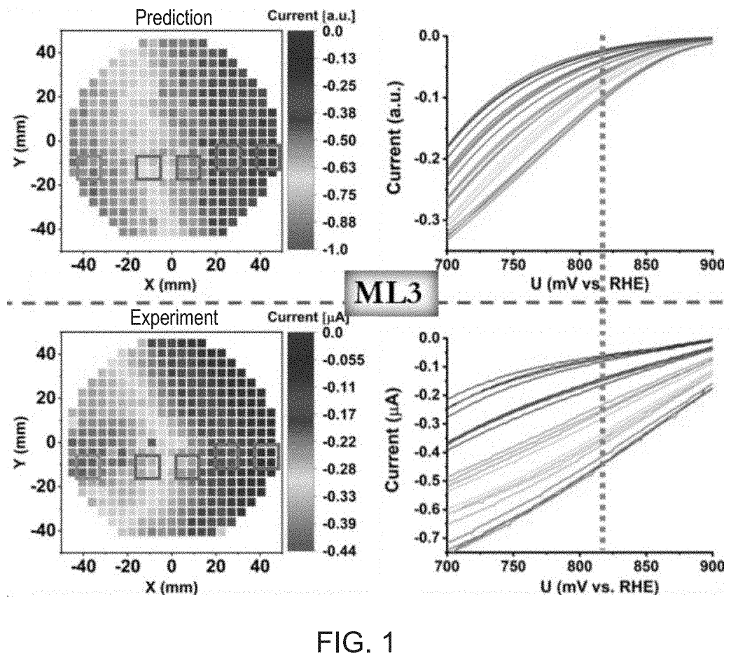

This framework has already been successfully deployed to predict electrocatalytic activity of multimetallic thin films in the oxygen reduction reaction (ORR) (FIG. 1).¹ However, in the case of thin films, surface segregation effects can be neglected and the surface composition at each pixel of the substrate can be considered to be identical to the bulk composition as deposited and, as such, is known. The situation is very different for discrete 3D complex nanomaterials. Multimetallic nanoparticles can form heterostructures with some constituent elements alloying with others phase separating. This makes it challenging to use such a framework with multimetallic nanoparticle electrocatalysts out-of-the-box without a robust and accurate strategy for predicting the surface structure. 1 Batchelor, T. A. A. et al. Complex-Solid-Solution Electrocatalyst Discovery by Computational Prediction and High-Throughput Experimentation**. Angew. Chem., Int. Ed. 60, 6932-6937 (2021).

One way to address this bottleneck is to reconstruct the surface structure of a catalyst by mapping corresponding predicted current densities to experimental measurements. That is, a possible surface configuration of unit cells for a catalyst of defined composition and size can be generated in silico and the corresponding current density can be calculated as described above. In the illustrative embodiment, the calculated current density is compared to the experimentally measured value and, if not within a prior-defined confidence interval, a new surface configuration of unit cells is generated, and a new theoretical current density value is calculated. This iterative process can be repeated until the calculated and experimental values are in agreement, at which point the mapping can be advanced to the next composition.

There are many computational and machine learning methods that can be deployed at this stage with varying efficiency and scalability. For example, the most probable surface configuration can be calculated using Monte Carlo simulations. The iterative optimization of the surface configuration to match the measurements can benefit from, e.g., reinforcement learning or generative adversarial network modeling. The latent space for the ML models can be designed to incorporate prior scientific knowledge and fundamental constraints.

Regardless of the combination of computational and machine learning methods being used, the goal can be to train a model that can accurately predict the distribution of active sites on the catalyst surface based on the composition, size, and experimental parameters without burdensome structural characterization. To achieve this ambitious goal, however, large and high-quality sets of training functional data may be necessary.

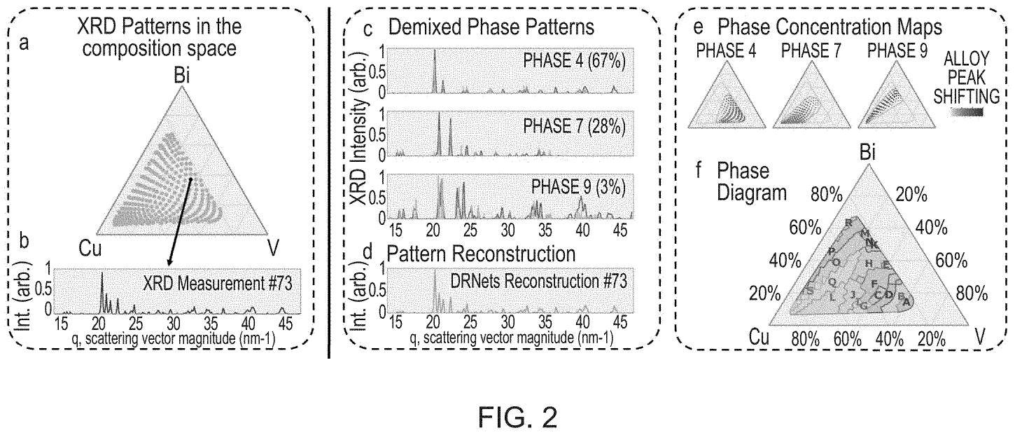

Constraint-bound ML for reconstructing composite data from the data for individual constituents combined in an unknown way is not necessarily new. For example, deep learning was previously combined with constraint reasoning to automate crystal-structure phase mapping that requires identifying crystal phases, or mixtures thereof, in X-ray diffraction measurements of synthesized materials (FIG. 2).² In this case, the X-ray diffraction pattern of a mixture of crystal phases is analogous to the surface configuration of a catalyst and individual crystal phases are analogous to the unit cells. 2 Chen, D. et al. Automating crystal-structure phase mapping by combining deep learning with constraint reasoning. Nature Machine Intelligence. 3, 812-822 (2021).

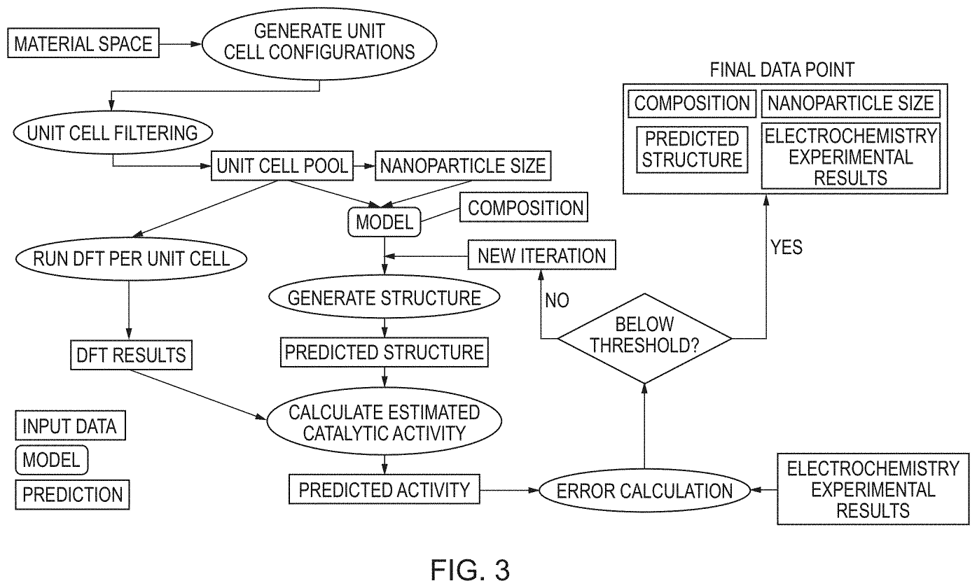

Although examples of certain piecemeal aspects of the proposed concept may already exist in the literature, at times in other fields; validating the feasibility of the idea, combining these aspects can be undertaken in a new and non-obvious way, i.e., calculating current densities for given unit cells using DFT and reconstructing complex catalyst surfaces of individual catalysts from unit cells in a similar way as solving the crystal-structure phase demixing problem. Furthermore, expanding the concept beyond just reconstructing the surface of a single catalyst can provide building a model that predicts the surface configuration of any catalyst within the design space. FIG. 3 provides a schematic of an example workflow.

As suggested in FIG. 3, an exemplary iterative workflow begins with a set of elements within the material space, labeled “Material Space.” The next step in the process utilizes the set of elements within the “Material Space” to “Generate Unit Cell Configurations,” containing the complete set of combinations of elements in the “Material Space.” All of the unit configurations generated can be subsequently filtered in the “Unit Cell Filtering” process, which can reduce the number of potential unit cells used in the iterative process and creates the “Unit Cell Pool.”

Density functional theory (DFT) simulations can be performed in the process labeled “Run DFT per Unit Cell” for each unit cell within the “Unit Cell Pool” to generate the “DFT Results,” containing the activity predictions for each unit cell. Next, the “Model” can take the inputs of the “Composition,” “Nanoparticle Size,” and “Unit Cell Pool” to generate an initial structure in the process block labeled “Generate Structures,” containing the unit-cell deterministic/probability ensemble structure labeled “Predicted Structure” for the given “Composition” and “Nanoparticle Size.”

The “Calculate Estimated Catalytic Activity” process block can sum the predicted activities from the “DFT Results” for each of the unit cells present in the “Predicted Structure” to calculate the total predicted activity, labeled “Predicted Activity,” of all the unit cells within the ensemble structure. The “Predicted Activity” can be compared against the “Electrochemistry Experimental Results” in the “Error Calculation” process to score the “Predicted Activity.” If this “Error Calculation” is above a specified threshold, another iteration can be performed to generate a new structure and repeat the evaluation. This workflow can continuously iterate until the “Error Calculation” is below the target threshold.

Once a satisfactory deterministic/probability structure is predicted within this workflow, the “Composition,” “Nanoparticle Size,” “Electrochemistry Experimental Results,” and “Predicted Structure” can be paired together as the “Final Data Point.” At this point, the workflow can begin again for the next nanoparticle within the “Material Space” and can be repeated until a desired training dataset for global machine learning model is generated. The global machine learning model, pre-trained on the generated dataset may minimize, or even completely eliminate, the need for additional computational modeling and/or workflow, as mentioned and suggested concerning FIG. 3, for predicting the (surface) structure of nanomaterials.

Although the workflow as mentioned and suggested concerning FIG. 3, at times, has been described within the context of nanoparticles and electrochemical activity, in some embodiments, this approach is translatable to other classes of nanomaterials, including nanofilms, nanosheets, core-shell nanostructures, quantum dots, halide perovskites, metal-organic frameworks, and other electronic, catalytic, optical and mechanical properties.

Within the present disclosure, electronic band structure of a material can provide information about its electronic properties and can be used to predict properties such as electrical conductivity, semiconducting behavior, and the presence of a band gap. The density of states can represent the number of electronic states available for electrons to occupy at a given energy level. Thus, insights can be provided into the electronic structure and help in predicting properties such as electrical conductivity and/or optical absorption can be achieved.

The charge carrier mobility can be an important property for electronic materials, as it influences the speed at which charge carriers (e.g., electrons or holes) can move through the material under an applied electric field. For magnetic materials, properties such as magnetic moments, exchange interactions, and/or magnetic anisotropy can be computed to understand and/or predict the material's magnetic behavior. Dielectric properties, such as the dielectric constant and/or loss tangent, can be important for materials used in capacitors, insulators, and/or other applications where electrical energy is stored and/or transmitted. Computationally assessed chemical stability can involve examining the material's reactivity and/or resistance to chemical degradation, corrosion, and/or oxidation. Adsorption energies associated with the adsorption of reactants onto catalyst surface can be important for evaluating the catalyst's activity. Strong adsorption can facilitate the breaking of reactant bonds, while weak adsorption can lead to poor catalytic performance. Computational models can be used to predict adsorption energies for various reactants on the catalyst surface. The electronic properties of a catalyst, such as the density of states and/or the position of the Fermi level, can influence its catalytic performance. For example, in heterogeneous catalysis, the electronic properties can affect the adsorption energies of reactants and/or the activation barriers of reaction steps.

Within the present disclosure, a deterministic approach to unit cell definition can include precision in selection of which unit cells are present on the surface at what ratio, e.g., each unit cell either present or not. Additionally or alternatively, a probabilistic approach to unit cell definition can include a likelihood profile that a particular unit cell or cells can be found on the surface within a certain probability, and in some embodiments, different unit cells may have different acceptable probabilities.

Concepts within the present disclosure can invert the current paradigm of going from structure to function, instead describing how large-scale functional data can be used to gain structural insights about materials. While designs within the present disclosure have been described concerning electrocatalysis as an example, this inverted paradigm can be applicable to any structure-function material relationships.

Within the present disclosure, functional screening methods may include: scanning electrochemical methods (scanning droplet cell, scanning electrochemical cell microscopy, scanning electrochemical microscopy), optical detection methods (fluorescence/phosphorescence turn-on/off, electrochromic detection), spatially isolated parallel experiments (microwell arrays, microelectrode arrays, parallel backed bed reactors), spectroscopic methods (scanning Raman, IR thermography, UV-VIS, (AT)-FTIR, high-throughput NMR), parallelized/scanning product collection (microfluidics, scanning droplets, capillary probes) with product analysis (mass spectroscopy, NMR, gas chromatography, liquid chromatography, IR, UV-VIS).

Data resulting from functional screening methods may include: current-potential traces, current density, onset potential, overpotential, optical images, fluorescence images, fluorescence intensity, transparency, color, conductivity, mass spectra, NMR spectra, Raman spectra, IR spectra, UV-VIS spectra, IR thermographs, product/reagent ratios, product conversion, turnover rates, byproduct formation rates, temperature. Data resulting from functional screening methods may be tied to one or several materials. Data resulting from functional screening methods may be tied to one specific location or area on a sample. Data resulting from functional screening methods may be measured over time.

The following ADDIN EN.REFLIST references are incorporated by reference in their entireties, and including at least those specific sections mentioned herein: Batchelor, T. A. A. et al. Complex-Solid-Solution Electrocatalyst Discovery by Computational Prediction and High-Throughput Experimentation**. Angew. Chem., Int. Ed. 60, 6932-6937 (2021); Chen, D. et al. Automating crystal-structure phase mapping by combining deep learning with constraint reasoning. Nature Machine Intelligence. 3, 812-822 (2021).

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

We claim:

1. A method of predicting material structural information based on functional characterization, the method comprising:
(a) representing a surface of a material as an ensemble of unit cells;
(b) determining a pool of possible unit cells based on one or more material input properties;
(c) computing functional characteristics of the unit cells within the pool;
(d) determining a combination of unit cells from the pool to represent a potential surface structure of the material and computing a corresponding cumulative functional characteristic for the material from the previously computed functional characteristics of individual unit cells of the combination;
(f) validating whether the computed cumulative functional characteristic matches at least one experimental measurement of the same functional property concerning the material;
repeating the steps (a)-(f), concerning a material surface of at least one other material to generate a dataset comprising the cumulative functional characteristic for each validated material, the dataset configured for training a global machine learning algorithm for predicting surface configuration of unit cells for still another material based on one or more material input properties of the still another material.

2. The method of claim 1, wherein computing functional characteristics includes at least one of: determining reactant adsorption energies and associated current densities related to electrocatalysis, and determining electronic properties related to optical or magnetic characteristics of materials.

3. The method of claim 1, wherein determining the functional characteristics of unit cells is based on the density functional theory (DFT) calculations exclusively, or as a combination of DFT with machine learning.

4. The method of claim 1, wherein determining the combination of unit cells to represent a potential surface structure is based on one or more of Monte Carlo simulations and a machine learning algorithm characterized as one or more of a deep learning model, a generative adversarial network (GAN), a transformer model, a reinforcement learning model, and an ensemble model.

5. The method of claim 4, wherein the machine learning model comprises one of random forest and genetic algorithm.

6. The method of claim 1, wherein the global machine learning algorithm for predicting the surface structure is based on one or more of a deep learning model, a generative adversarial network (GAN), a transformer model, a reinforcement learning model, and an ensemble model.

7. The method of claim 6, wherein the global machine learning model comprises one of random forest and genetic algorithm.

8. The method of claim 1, wherein validating whether the computed functional property matches the at least one experimental measurement includes determining whether a prediction threshold is achieved.

9. The method of claim 8, wherein determining whether the prediction threshold is achieved includes determining whether difference between the computed functional property and the experimentally measured functional properties is within a predetermined range of values.

10. The method of claim 1, wherein validating includes determining that the computed functional property does not match the experimental measurements concerning the material surface of the material, and reiterating steps (a)-(f) until the potential surface structure yielding the computed functional property matches the at least one experimental measurement.

11. The method of claim 1, wherein configuration for training a machine learning algorithm does not require conducting steps (i)-(e) for the still another material.

12. The method of claim 1, wherein the one or more material input properties of the material is defined only as composition of the material.

13. The method of claim 1, wherein the one or more material input properties of the still another material is defined only as composition of the still another material.

14. The method of claim 1, wherein determining the combination of unit cells includes predicting the potential surface structure as a deterministic ensemble of unit cells.

15. The method of claim 1, wherein determining the combination of unit cells includes predicting the potential surface structure as a probabilistic ensemble of unit cells.

16. A system comprising: at least one processor executing instructions stored in memory for conducting the method of claim 1.

17. A method of predicting material structural information in relation to functional characterization, the method comprising:
determining functional characteristics of units cells among a pool of unit cells of a material surface;
determining a predicted structure of the material surface based on the unit cell pool; and
determining a predicted material activity based on the determined functional characteristics and the determined predicted structure, and outputting a characterization of material structural information of the material surface based on the predicted material activity.

18. The method of claim 17, wherein determining the predicted material activity includes determining whether a threshold prediction of material activity is achieved, and re-determining the predicted structure in response to determination that the threshold prediction has not be achieved.

19. The method of claim 18, wherein the threshold prediction of material activity is determined by comparison of the predicted material activity with experimental results.

20. The method of claim 18, wherein in response to determination that the threshold prediction is achieved, outputting is performed.

21. The method of claim 17, wherein outputting a characterization includes a dataset for training a machine learning model for predicting the surface configuration of unit cells for new materials based on material input properties.
